(19) United States
(12) Patent Application Publication
(10) Pub. No.: US 2015/0230868 A1
(43) Pub. Date: Aug. 20, 2015

(54) Needle Guide For Percutaneous Lung Biopsy

(71) Applicant: JJ Dogs LLC, Indianapolis, IN (US)

(72) Inventor: Sean Miller, Greenwood, IN (US)

(21) Appl. No.: 14/625,230

(22) Filed: Feb. 18, 2015

Related U.S. Application Data
(60) Provisional application No. 61/941,115, filed on Feb. 18, 2014.

(51) Int. Cl. A61B 19/00; A61B 17/34; A61B 10/02; A61B 10/04

(52) U.S. Cl. CPC A61B 19/201; A61B 10/04; A61B 17/3403; A61B 10/0283; A61B 2010/045; A61B 2017/00862

(57) ABSTRACT

A needle guide for use in percutaneous lung biopsy procedures includes a cup that can be affixed in air-tight engagement to the patient's skin around the incision. The cup includes a hub adapted to receive an introducer or biopsy needle in sealed engagement as the needle is advanced into the incision. The cup may be transparent to allow visualization of the incision to facilitate navigation of the needle into the incision. The needle guide is further provided with a fluid tube for engagement with a syringe operable to draw a slight vacuum within the cup to thereby prevent infiltration of air into the incision during the biopsy procedure.

### PRIORITY CLAIM

This application claims priority to Provisional U.S. Patent Application No. 61/941,115, filed on Feb. 18, 2014, the disclosure of which is incorporated herein by reference in its entirety.

### BACKGROUND

A lung biopsy is a procedure performed to obtain samples of lung tissue, or of other tissue that is within the lung, to be viewed under a microscope. Typically, the samples are obtained and viewed to facilitate diagnosis of lung disease or cancer. Lung biopsies are performed using either an open method or a closed method. In an open method biopsy, a general anesthesia is used to anesthetize the patient, and surgical incisions are made in the chest and lungs to remove the target tissue. In a closed method biopsy, a local anesthetic can be used and a small instrument is inserted through an incision made in the chest to remove the target tissue. The incisions made in a closed method biopsy are much smaller than made in an open method biopsy because the instrument used in a closed method biopsy is smaller than that used in an open method biopsy. Accordingly, closed method biopsies are less invasive procedures and are preferred when possible.

One closed method biopsy procedure is a percutaneous needle biopsy as generally depicted in FIG. 1. Percutaneous lung biopsies can be performed, for example, to obtain samples of tissue that are located close to the patient's chest wall. In this procedure, the location of the target tissue is verified using an imaging technique, such as an X-ray or CT scan. Once the location of the target tissue is verified, an insertion site is marked on the patient's skin where a biopsy needle, which will be directed to the location of the target tissue, will be inserted. To insert the biopsy needle, the insertion site is locally anaesthetized, an insertion incision is made in the patient's chest wall at the mark on the patient's skin, and a biopsy needle is guided through the chest wall, via the insertion incision. Once the biopsy needle has been inserted, imaging equipment may be used to guide the tip of the biopsy needle to the target tissue. At the location of the target tissue, the tip of the biopsy needle obtains a sample of lung tissue, then the biopsy needle is retracted and the insertion incision is closed.

One challenge which arises in the performance of percutaneous lung biopsies is proper placement of the biopsy needle for insertion into and through the patient's chest wall and the pleura of the patient's lung to the target tissue. This challenge is exacerbated by the patient's breathing and by deflection of the chest wall due to the patient's heartbeat, which can alter the location of the biopsy needle. When imaging equipment is used to guide the biopsy needle within the patient's tissue, an introducer needle is often required to accurately locate the target tissue site.

Another challenge which arises in the performance of percutaneous lung biopsies is the infiltration of air into the space between the lung pleura and the inner wall of the chest. This air can lead to a pneumothorax, or collapsed lung, by pressing against the outside of the patient's lung. A larger, more serious pneumothorax may require additional medical procedures to correct.

### SUMMARY

A guide for guiding a biopsy needle at an insertion site includes a body and a hub. The body is configured to be arranged at the insertion site and includes a perimeter and a lip formed along the perimeter. The hub projects from the body and is spaced apart from the perimeter. The hub includes a flexible member configured to pass the biopsy needle therethrough and a passage configured to pass air from a first side of the hub to a second side of the hub.

A needle guide for use at a surgical insertion site includes a body and a biopsy needle. The body is configured to be removably coupled to the surgical insertion site, and the biopsy needle is configured to be partially inserted through the body and into the surgical insertion site. The body is configured to form an airtight seal at the surgical insertion site when the body is coupled to the surgical insertion site. The biopsy needle and the body are configured to form an airtight seal between the biopsy needle and the body when the at least a portion of the biopsy needle is passed through the body. The body includes a passage configured to pass air from a first side of the body, arranged toward the surgical insertion site, to a second side of the body, arranged away from the surgical insertion site.

### DESCRIPTION OF THE FIGURES

FIG. 1 is a pictorial representation of a percutaneous lung biopsy procedure.

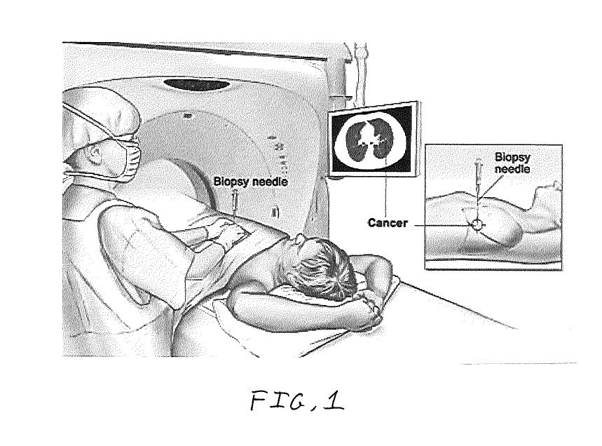

FIG. 2 is a perspective view of a needle guide according to one aspect of the present disclosure.

FIG. 3 is a side partial cross-sectional view of the needle guide shown in FIG. 2.

FIG. 4 is a bottom view of the needle guide shown in FIG. 2.

FIG. 5 is a top view of the needle guide shown in FIG. 2.

### DETAILED DESCRIPTION

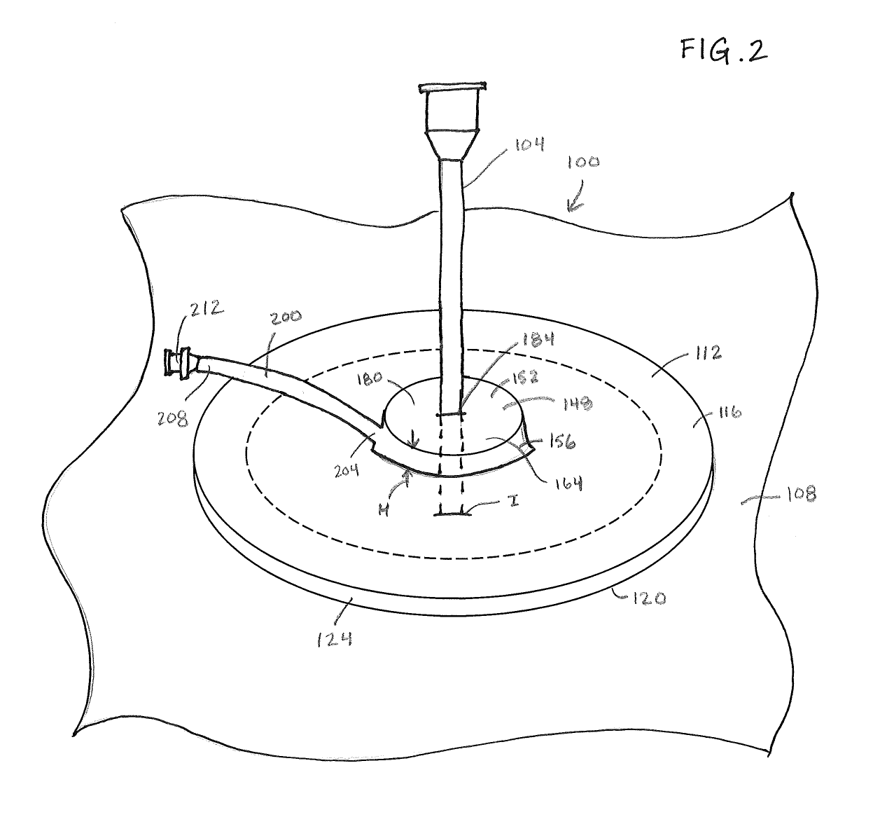

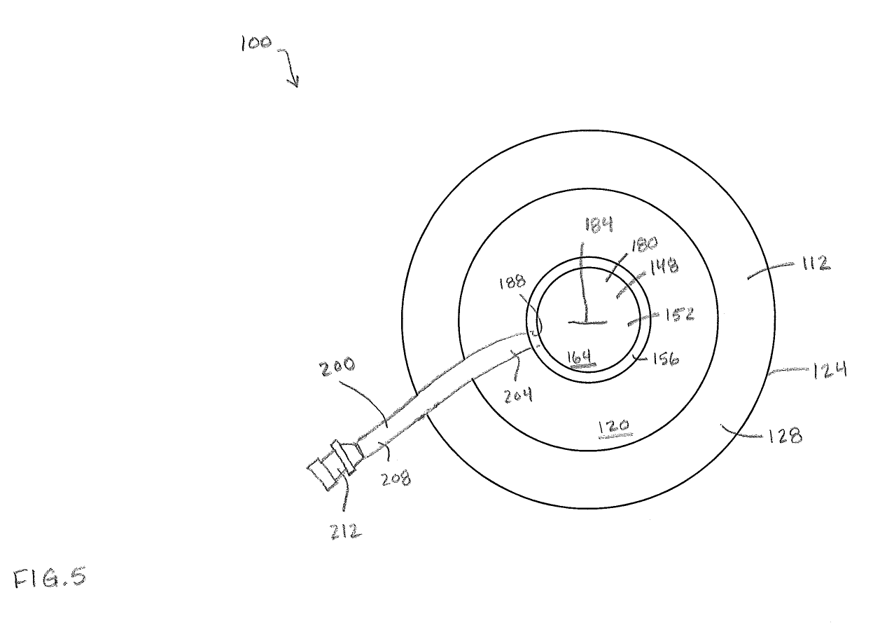

In order to address the problems described above for percutaneous lung biopsies, the present disclosure provides a needle guide 100, as illustrated in FIGS. 2-5, for guiding a biopsy needle 104 at an insertion site 108. The needle guide 100 includes a body 112 that is removably coupled to the patient's skin at the insertion site 108 such that the biopsy needle 104 inserted through the needle guide 100 is guided by the needle guide 100 to be aligned with a surgical incision I formed in the patient's skin at the insertion site 108. The body 112 of the needle guide 100 is sized to encircle and surround the incision I made at the insertion site 108. In one specific embodiment, the body 112 can have a diameter of between approximately 4 cm and approximately 5 cm. The body 112 is formed of a material that is at least translucent and preferably substantially transparent to allow visualization of the incision I when the body 112 of the needle guide 100 is placed on the patient's skin at the insertion site 108. The material of the body 112 is also sufficiently flexible to conform to the surface of the patient's skin at the insertion site 108 but rigid enough to maintain its shape even under a slight vacuum. In one specific embodiment the body 112 is formed of a clear silicone.

The body 112 includes a first body side 116 arranged facing toward the insertion site 108 when the body 112 is coupled to the patient's skin at the insertion site 108, a second body side 120 arranged facing away from the insertion site 108 when the body 112 is coupled to the patient's skin at the insertion site 108, and a perimeter 124 formed around the body 112 where the first body side 116 and the second body side 120 meet. The body 112 also includes a lip 128 formed as a rim along the perimeter 124 of the body 112 and adapted to be removably coupled to the patient's skin surrounding the incision I. In other words, the body 112 is coupled to the patient's skin via the lip 128. In at least one embodiment, the body 112 is substantially circularly shaped such that the perimeter 124 is also a circumference of the body 112 and the lip 128 is formed as an annular rim. However, the body 112 need not be substantially circularly shaped.

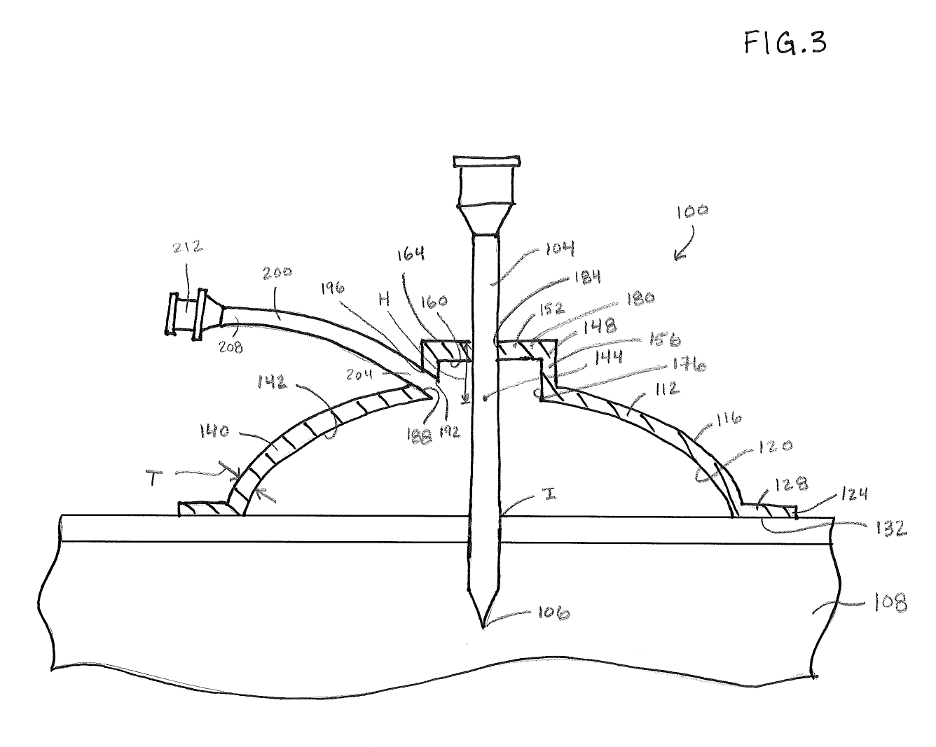

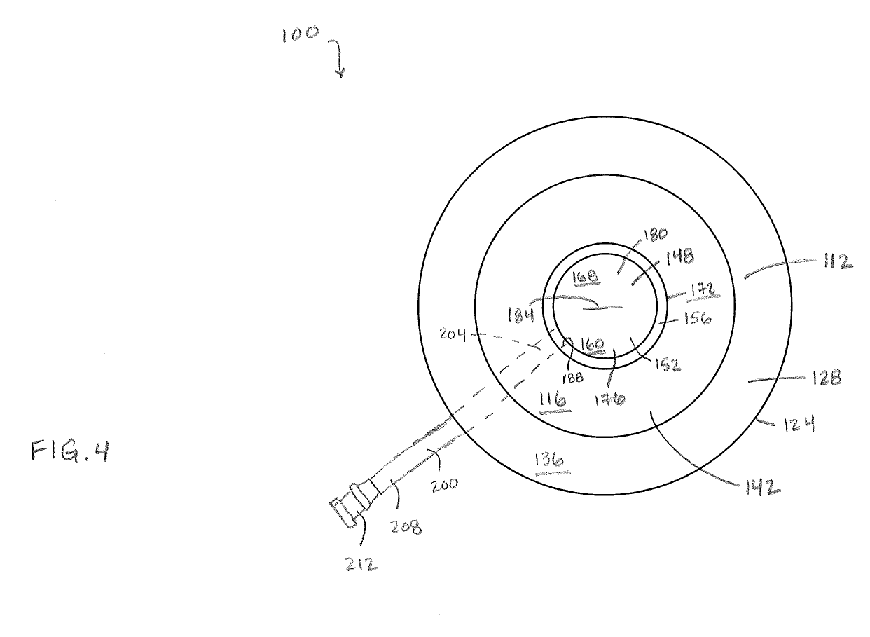

In at least one embodiment, the lip 128 is removably coupled to the patient's skin by an adhesive coating 132 (shown in FIG. 3) applied to the lip 128. In one embodiment, the adhesive coating 132 is covered by a removable covering 136, such as a removable backing, as shown in FIG. 4. The removable covering 136 covers the adhesive coating 132 until the guide 100 is to be used. The removable covering 136 is then removed to expose the adhesive coating 132 so that the body 112 can be adhered to the patient's skin.

The adhesive coating 132 is compatible with skin tissue and capable of ready adherence but easy removal. Additionally, the adhesive coating 132 is adapted to provide a uniform seal around the lip 128 that is at least sufficient to withstand a slight vacuum applied between the body 112 and the insertion site 108 during the biopsy procedure. In one embodiment, the adhesive coating 132 may be a latex-based or a silicone-based adhesive that may require an adhesive remover composition in order to remove the adhesive coating 132 from the patient's skin. In another embodiment, the adhesive coating 132 may be a polyurethane adhesive, such as the adhesive used on bandages, which does not require a separate adhesive remover to remove the adhesive coating 132 from the patient's skin.

The body 112 further includes a wall 140 that provides some structural rigidity and shape to the body 112. In at least one embodiment, the wall 140 provides the body 112 with a dome shape within the lip 128 such that the first body side 116 is generally concave and the second body side 120 is generally convex. In an alternative embodiment, the wall 140 does not provide the body 112 with a dome shape and the lip 128 extends along most of the wall 140 of the body 112. Regardless of the shape of the body 112 provided by the wall 140, when the body 112 is coupled to the patient's skin at the insertion site 108, the wall 140 forms an air space or a cavity 142 between the first body side 116 and the patient's skin. The wall 140 preferably has a wall thickness T (shown in FIG. 3) sufficient to retain the shape of the body 112 even when a slight vacuum is drawn in the cavity 142 between the first body side 116 and the patient's skin at the insertion site 108.

The wall 140 of the body 112 defines a center 144 (shown in FIG. 3) located substantially at the center of the body 112. In embodiments where the body 112 is substantially circularly shaped, the center 144 is equidistant from the entire perimeter 124 of the body 112. The guide 100 further includes a barrel or hub 148 which projects from the second body side 120 of the body 112 at the center 144 of the wall 140. The hub 148 is substantially shaped as a hollow cylinder having a planar wall 152 that is spaced apart from the wall 140 of the body 112 and a transverse wall 156 extending between the planar wall 152 and the body 112. The transverse wall 156 is substantially perpendicular to the planar wall 152. In embodiments where the body 112 is substantially circularly shaped, the hub 148 is concentrically disposed on the body 112 within the perimeter 124 and around the center 144 such that the transverse wall 156 is concentric with the perimeter 124. In alternative embodiments, the hub 148 need not be substantially cylindrically shaped. The insertion site 108 is substantially planar. When the body 112 of the guide 100 is coupled to the patient's skin, the planar wall 152 is arranged substantially parallel with the insertion site 108. Accordingly, when the body 112 of the guide 100 is coupled to the patient's skin, the transverse wall 156 is substantially perpendicular to the insertion site 108.

The hub 148 has a first hub side 160 (shown in FIG. 3 and FIG. 4) facing toward the body 112 and a second hub side 164 facing away from the body 112. The planar wall 152 includes a planar surface 168 (shown in FIG. 4) and the transverse wall 156 includes a transverse surface 172 (shown in FIG. 4), each of which are arranged on the second hub side 160 facing away from the body 112. Within the first hub side 160 the hub 148 defines a hub cavity 176 that is in fluid communication with the cavity 142 of the body 112. Accordingly, when a vacuum is drawn within the cavity 142 of the body 112, a vacuum is also drawn within the hub cavity 176.

The hub 148 further includes a flexible member 180 having a slit 184 configured to pass at least a portion of the biopsy needle 104 therethrough. In at least one embodiment, the flexible member 180 is provided as the planar wall 152 of the hub 148. The flexible member 180 is formed such that the slit 184 forms an airtight seal of the hub 148. The slit 184 is formed such that, when the portion of the biopsy needle 104 is inserted therethrough, an airtight seal is formed between the hub 148 and the biopsy needle 104. In at least one embodiment, the biopsy needle 104 can be formed to contribute to and facilitate the formation of the airtight seal between the hub 148 and the biopsy needle 104. The slit 184 is further formed such that, when the biopsy needle 104 is removed from the slit 184, the slit 184 once again forms an airtight seal of the hub 148.

When the portion of the biopsy needle 104 is inserted through the slit 184, a tip 106 of the biopsy needle 104 is passed through the hub cavity 176, through the cavity 142 of the body 112 and into the incision I formed in the patient's skin at the insertion site 108. To this end, the slit 184 should be substantially aligned with the incision I when the body 112 is coupled to the patient's skin at the insertion site 108. The substantially clear material used to form the body 112 facilitates this alignment. Additionally, the flexible member 180 is flexible enough to enable the biopsy needle 104 to be slightly manipulated, if necessary, for insertion into the incision I if the slit 184 is not aligned directly above the incision I.

The flexible member 180 is also rigid enough to support the biopsy needle 104 in an upright position when the portion of the biopsy needle 104 is inserted through the slit 184. When the biopsy needle 104 is in the upright position, a longitudinal axis 110 of the biopsy needle 104 is substantially perpendicular to the flexible member 180. In embodiments where the flexible member 180 is provided as the planar wall 152 of the hub 148, when the biopsy needle 104 is in the upright position, a longitudinal axis 110 of the biopsy needle 104 is substantially perpendicular to the planar wall 152 of the hub 148. The flexible member 180 can be, for example, a flexible membrane formed of, for example, silicone.

The flexible member 180 and the slit 184 are preferably sized to accept a conventional introducer or biopsy needle 104. The transverse wall 156 of the hub 148 can have a height H (shown in FIG. 2 and FIG. 3) such that the planar wall 156 is spaced above the above the body 112. In embodiments where the flexible member 180 is provided as the planar wall 156, the height H can provide support for an introducer or biopsy needle 104 that has been inserted through the slit 184 and into the incision I. In one specific embodiment, the transverse wall 156 can have a height H of between approximately 0.5 cm and approximately 1.0 cm.

The hub 148 further includes a passage 188 extending through the transverse wall 156 from the first hub side 160 to the second hub side 164. The passage 188 is configured to pass air from within the hub cavity 176 and the cavity 142 of the body 112 to outside of the hub 148 and the body 112. To this end, the passage 188 includes a first passage end 192 (shown in FIG. 3) arranged on the first hub side 160 and a second passage end 196 (shown in FIG. 3) arranged on the second hub side 164. In an alternative embodiment, the passage 188 can be formed partially through the transverse wall 156 of the hub 148 and partially through the wall 140 of the body 112.

The passage 188 includes a flexible tube 200 coupled to the hub 148 so as to project from the second passage end 196. In one embodiment, the flexible tube 200 is integrally formed with the passage 188, and thus is integrally formed with the hub 148. In another embodiment, the flexible tube 200 is separate from the passage 188 and the hub 148 and is inserted into the second passage end 196. In both embodiments, the flexible tube 200 is in fluid communication with the hub cavity 176 and the cavity 142 of the body 112 and forms an airtight seal with the passage 188 and the hub 148. In embodiments where the passage 188 is partially formed in the body 112, the flexible tube 200 also forms an airtight seal with the body 112.

The flexible tube 200 includes a first tube end 204 integrally formed with or coupled to the passage 188 and a second tube end 208 opposite the first tube end 204. The flexible tube 200 further includes a fluid-tight fitting 212 at the second tube end 208. The fluid-tight fitting 212 can be, for example, a Luer fitting. The fluid-tight fitting 212 is also formed as a vacuum locking fitting configured for engagement to a vacuum source (not shown). The vacuum source, which can preferably be a syringe, is coupled to the fluid-tight fitting 212 and is then used to draw a slight vacuum through the flexible tube 200. Accordingly, the vacuum source can draw a slight vacuum in the cavity 142 of the body 112 and the hub cavity 176 via the passage 188 and the flexible tube 200.

In use, the needle guide 100 is centered on the incision I at the insertion site 108, as visualized through the body 112. The removable covering 136 is removed from the lip 128 to expose the adhesive coating 132 to enable removably adhering the body 112 to the patient's skin by removably adhering the lip 128 at the insertion site 108. The removable covering 136 may be provided in multiple segments so that the segments can be individually removed and discrete sections of the lip 128 affixed to the patient's skin. Once the lip 128 around the entire perimeter 124 of the body 112 has been affixed to the patient's skin, a vacuum can be drawn in the cavity 142 of the body 112 and the hub cavity 176 by a vacuum source coupled to the fluid-tight fitting 212 of the flexible tube 200. The vacuum can be periodically maintained by the vacuum source during the biopsy procedure to ensure that air cannot pass into the chest of the patient through the incision I.

Once the body 112 is affixed to the patient's skin, an introducer or the biopsy needle 104 can be advanced through the slit 184 formed in the flexible member 180 of the hub 148 and into the incision I. The introducer or the biopsy needle 104 may incorporate an additional seal that bears against the transverse surface 172 of the hub 148 when the introducer or biopsy needle 104 is in position within the incision I. With the introducer or biopsy needle 104 in position adjacent the target tissue the vacuum source can again be used to pull a slight vacuum in the cavity 142 of the body 112 and the hub cavity 176 through the flexible tube 200 to ensure that the cavity 142 and the hub cavity 176 are again air-tight. In the case where an introducer is used, a biopsy needle 104 may then be advanced through the introducer to obtain the biopsy sample. It can be appreciated that the airtight seal formed at the slit 184 allows the position of the introducer and/or biopsy needle 104 to be set and adjusted multiple times without risk of air infiltration. The vacuum source can be used to draw a vacuum with each adjustment. Once the sample has been obtained, the biopsy needle 104 and introducer can be removed. The vacuum source can be disconnected from the flexible tube 200 to allow air to enter the cavity 142 and the hub cavity 176. The body 112 can then be removed from the patient's skin, with the use of a skin-safe adhesive solvent if necessary.

In an alternative use, the flexible tube 200 can be used to introduce a skin-safe injectable sealant into the cavity 142. The sealant may be an inert foam that fills the cavity 142 to prevent air from entering the incision I. The sealant may also incorporate an antibiotic composition. Once the foam has been introduced, the flexible tube 200 may be connected to a positive pressure source (not shown) that is operated to slightly pressurize the cavity 142 in order to effectively push the foam into the incision I around the biopsy needle 104. Afterward, additional sealant may be added to fill the cavity 142.

In one embodiment, the needle guide 100 includes the biopsy needle 104, the body 112, and the hub 148. In another embodiment, the needle guide 100 includes only the body 112 and the hub 148. In one embodiment, the body 112 includes the hub 148.

What is claimed is:

1. A guide for guiding a biopsy needle at an insertion site, comprising:
a body configured to be arranged at the insertion site, the body including;
a first body side arranged toward the insertion site and a second body side arranged away from the insertion site;
a perimeter formed where the first body side and the second body side meet; and
a lip formed in the first body side along the perimeter and configured to be removably coupled to the insertion site, the body configured to form an air space within the perimeter and between the first body side and the insertion site when the lip of the body is coupled to the insertion site; and
a hub projecting from the second body side and spaced apart from the perimeter, the hub including;
a flexible member configured to pass the biopsy needle therethrough; and
a passage fluidly connected to the air space and configured to pass a fluid through the hub.

2. The guide as claimed in claim 1, wherein the body is silicone.

3. The guide as claimed in claim 1, wherein:
the passage includes a flexible tube integrally formed with the hub and in fluid communication with the air space.

4. The guide as claimed in claim 1, wherein:
the passage includes a flexible tube,
the passage includes an opening extending through the hub from the first side of the hub to the second side of the hub, and
the opening is configured to receive the flexible tube on the second side of the hub.

5. The guide as claimed in claim 1, wherein:
the flexible member is a flexible membrane including a slit configured to pass a portion of the biopsy needle therethrough.

6. The guide as claimed in claim 5, wherein:
the flexible membrane forms an airtight seal with the portion of the biopsy needle when the portion of the biopsy needle is passed through the slit.

7. The guide as claimed in claim 1, wherein the lip of the body includes an adhesive configured to removably couple the body to the insertion site.

8. The guide as claimed in claim 7, further comprising a removable covering coupled to the lip of the body such that the removable covering covers the adhesive until removed.

9. The guide as claimed in claim 1, wherein:
the passage includes a flexible tube having a first tube end arranged at the hub and in fluid communication with the air space and a second tube end arranged away from the hub, and
the flexible tube includes a fluid-tight fitting at the second tube end.

10. The guide as claimed in claim 9, wherein the fluid-tight fitting is configured to be coupled to a vacuum source such that operation of the vacuum source removes air from the air space via the passage and the flexible tube.

11. The guide as claimed in claim 1, wherein the body and the hub are formed from a substantially transparent material.

12. The guide as claimed in 1, wherein:
the body forms an airtight seal with the insertion site when the lip of the body is coupled to the insertion site.

13. The guide as claimed in claim 1, wherein:
the body forms a dome shape above the insertion site when the lip of the body is coupled to the insertion site.

14. The guide as claimed in claim 1, wherein:
the hub includes a planar surface on the second side of the hub, and
the flexible member is configured to support the biopsy needle in a position such that a longitudinal axis of the biopsy needle is substantially perpendicular to the planar surface of the hub.

15. The guide as claimed in claim 14, wherein:
the insertion site is substantially planar, and
the planar surface of the hub is substantially parallel to the insertion site when the lip of the body is coupled to the insertion site.

16. The guide as claimed in claim 14, wherein:
the hub includes a transverse wall extending between the planar surface of the hub and the body, and
the passage is at least partially formed in the transverse wall of the hub.

17. A needle guide for use at a surgical insertion site, comprising:
a body having a lip configured to be removably coupled to the surgical insertion site; and
a biopsy needle configured to be partially inserted through the body and into the surgical insertion site, wherein
the body is configured to form an airtight seal at the surgical insertion site when the lip is coupled to the surgical insertion site,
the biopsy needle and the body are configured to form an airtight seal between the biopsy needle and the body when the at least a portion of the biopsy needle is passed through the body, and
the body includes a passage configured to pass air from a first side of the body, arranged toward the surgical insertion site, to a second side of the body, arranged away from the surgical insertion site.

18. The needle guide as claimed in claim 17, wherein:
the passage includes a flexible tube having a first tube end in fluid communication with the first side of the body and a second tube end, opposite the first tube end, including a fluid-tight fitting configured to be coupled to a vacuum source.

19. The needle guide as claimed in claim 18, wherein:
the body is configured to form an air space between the first side of the body and the surgical insertion site when the lip is coupled to the surgical insertion site, and
the fluid-tight fitting is configured to be coupled to a vacuum source such that operation of the vacuum source removes air from the air space via the passage and the flexible tube.

20. The needle guide as claimed in claim 19, wherein:
the lip is formed along a perimeter of the body, and
the air space is formed within the perimeter.
